(19) United States
(12) Patent Application Publication
(10) Pub. No.: US 2022/0008392 A1
(43) Pub. Date: Jan. 13, 2022

(54) METHODS OF TREATING CANCER

(71) Applicant: ASTRAZENECA AB, Södertälje (SE)

(72) Inventors: Justin CIDADO, Wilmington, DE (US); Scott BOIKO, Wilmington, DE (US); Lisa DREW, Wilmington, DE (US)

(21) Appl. No.: 17/293,155

(22) PCT Filed: Nov. 13, 2019
(86) PCT No.: PCT/EP2019/081145

§ 371 Date: May 12, 2021

Related U.S. Application Data
(60) Provisional application No. 62/767,215, filed on Nov. 14, 2018.

(51) Int. Cl. A61K 31/4162; A61K 31/4439; G01N 33/574

(52) U.S. Cl. CPC A61K 31/4162; G01N 2333/82; G01N 33/57426; A61K 31/4439

(57) ABSTRACT

Methods of treating Mcl-1 dependent cancers are described herein. The methods can include determining whether the cancer is Bfl-1 positive, and administering an inhibitor of CDK9 to a patient if the cancer is Bfl-1 positive.

Myeloid Cell Leukemia 1 (Mcl-1) is an important anti-apoptotic member of the BCL-2 family of proteins and a master regulator of cell survival. Amplification of the MCL1 gene and/or overexpression of the Mcl-1 protein has been observed in multiple cancer types and is commonly implicated in tumor development. In fact, MCL1 is one of the most frequently amplified genes in human cancer. In many malignancies, Mcl-1 is a critical survival factor and it has been shown to mediate drug resistance to a variety of anti-cancer agents.

Mcl-1 promotes cell survival by binding to pro-apoptotic proteins like Bim, Noxa, Bak, and Bax and neutralizing their death-inducing activities. Inhibition of Mcl-1 thereby releases these pro-apoptotic proteins, often leading to the induction of apoptosis in tumor cells dependent on Mcl-1 for survival.

Like Mcl-1, Bfl-1 also belongs to the BCL-2 family of anti-apoptotic proteins.

Cyclin-dependent protein kinases (CDKs) represent a family of serine/threonine protein kinases that become active upon binding to a cyclin regulatory partner. CDK/cyclin complexes were first identified as regulators of cell cycle progression. CDK/cyclin complexes have also been implicated in transcription and mRNA processing. CDK9/PTEFb (positive transcription elongation factor b) phosphorylates the carboxyl-terminal domain (CTD) of the large subunit of RNA polymerase II (RNAP II), predominantly at Ser-2, regulating elongation of transcription. Inhibition of CDK9 and transcriptional repression results in the rapid depletion of short lived mRNA transcripts and associated proteins, including Mcl-1 and c-myc, leading to induction of apoptosis in cancer cells that are hyper-dependent on these survival proteins.

There is a need for methods to determine which cancers, and thereby which patients, are susceptible to treatments that alter levels of anti-apoptotic proteins such as, for example, Mcl-1 and Bfl-1.

In one aspect, a method of treating an Mcl-1 dependent cancer in a patient is provided, which includes determining whether the cancer is Bfl-1 positive by obtaining or having obtained a biological sample from the patient; and performing or having performed an assay to measure the expression level of Bfl-1; and if the cancer is Bfl-1 positive, then administering an inhibitor of CDK9 to the patient, thereby increasing cancer apoptosis; wherein the increase in cancer apoptosis for a Bfl-1 positive cancer upon administration of an inhibitor of CDK9 is greater than it would be in a Bfl-1 negative cancer, and/or the increase in cancer apoptosis for a Bfl-1 positive cancer upon administration of an Mcl-1 inhibitor is less than it would be in a Bfl-1 negative cancer.

In another aspect, the use of an inhibitor of CDK9 in the treatment of an Mcl-1 dependent cancer is provided, wherein the cancer has been determined to be Bfl-1 positive.

In another aspect, the use of an Mcl-1 inhibitor in the treatment of an Mcl-1 dependent cancer is provided, wherein the cancer has been determined to be Bfl-1 negative.

Other features, objects, and advantages will be apparent from the description and drawings, and from the claims.

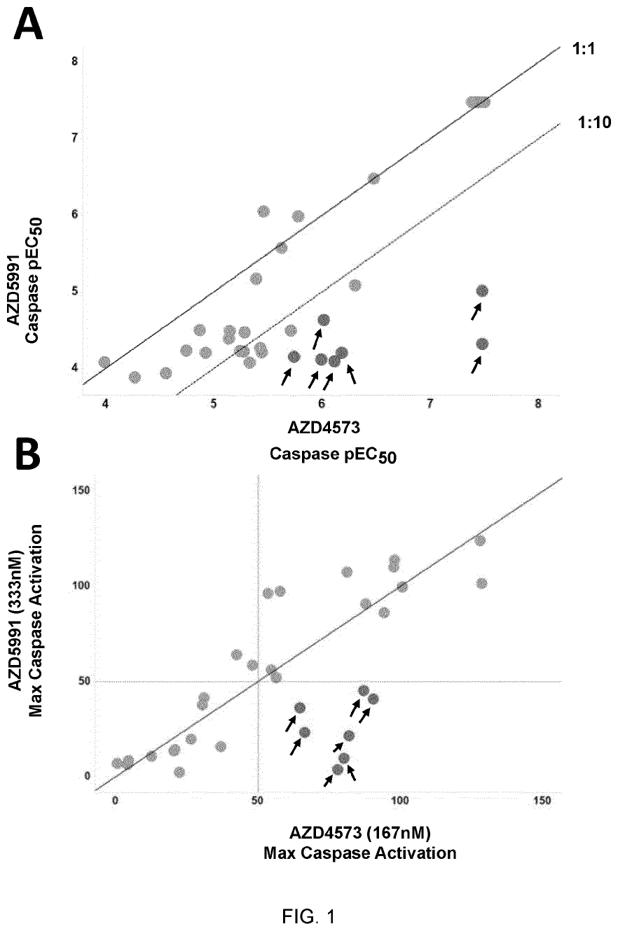

FIGS. 1A-1B illustrate differential response between CDK9 inhibition compared to Mcl-1 inhibition for some lymphoma cell lines.

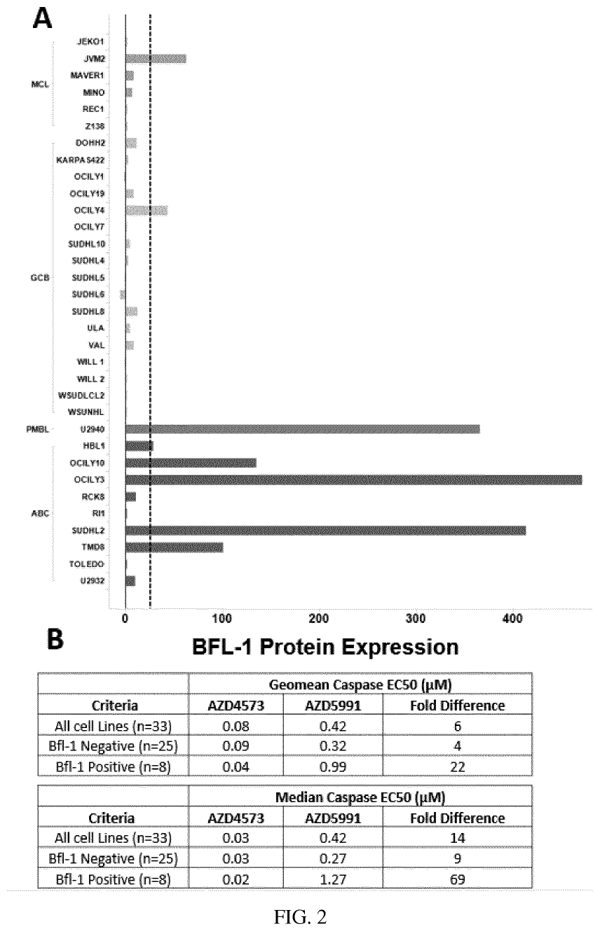

FIG. 2 shows that Bfl-1 expression is associated with relatively greater sensitivity to CDK9 inhibition compared to sensitivity to Mcl-1 inhibition.

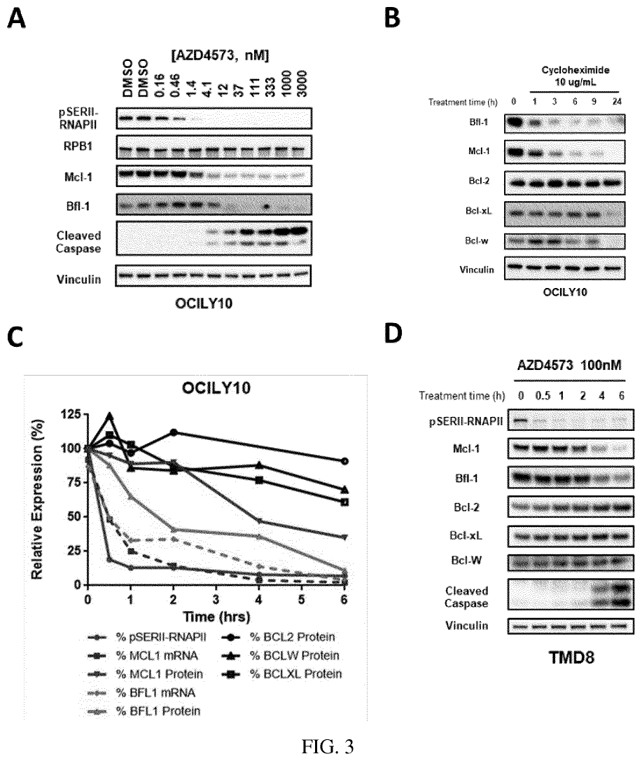

FIGS. 3A-3D show that Bfl-1 is a labile protein, and its expression is modulated by transient CDK9 inhibition.

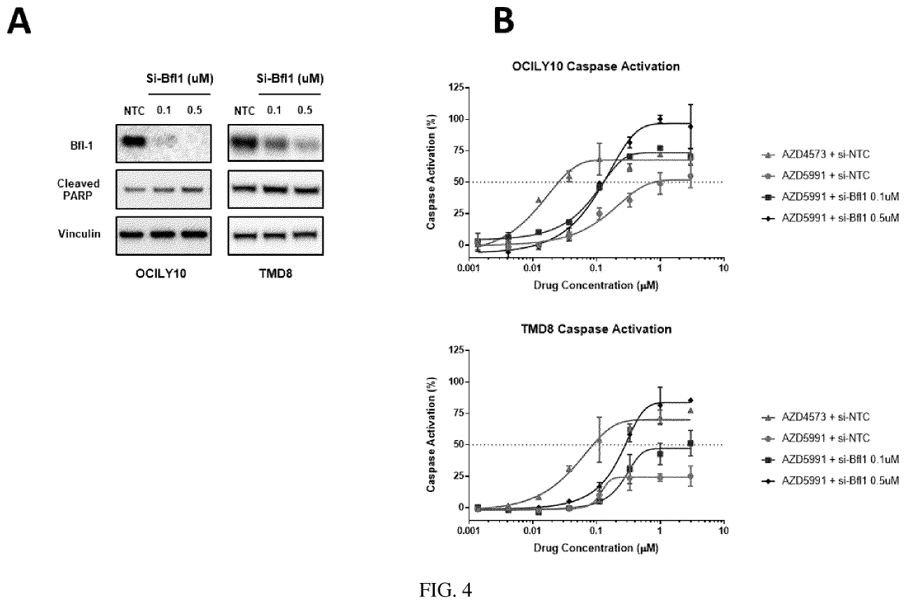

FIGS. 4A-4B show that Bfl-1 expressing lymphoma cell lines depend on multiple Bcl-2 family proteins for survival.

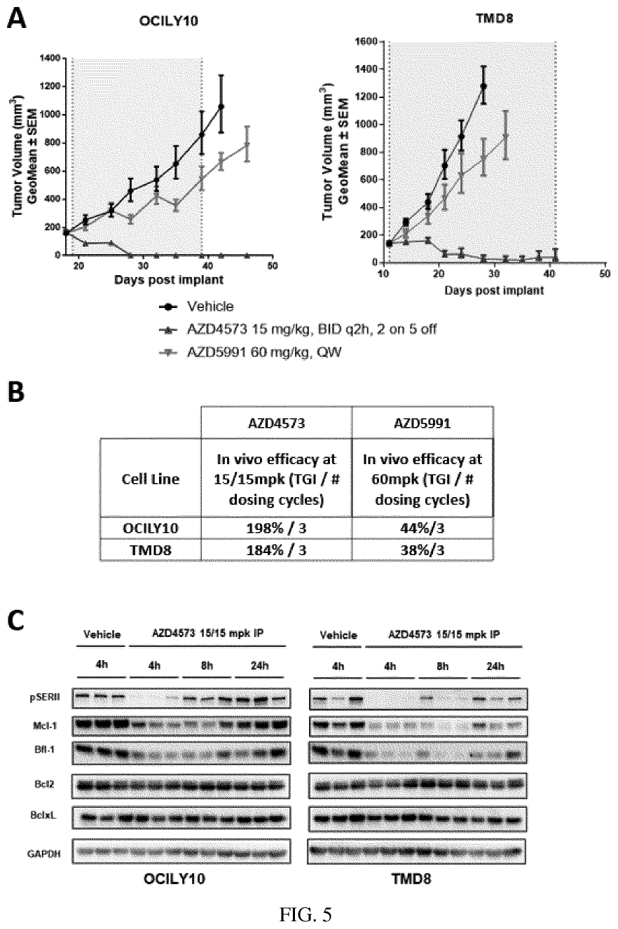

FIGS. 5A-5C show that AZD4573 demonstrates robust anti-tumor activity in ABC-DLBCL cell lines.

Described herein are methods of treating Mcl-1 dependent cancers. The methods generally involve identifying those Mcl-1 dependent cancers that have increased sensitivity to CDK9 inhibition and/or decreased sensitivity to Mcl-1 inhibition, as compared to other Mcl-1 dependent cancers.

Without intending to be bound by a particular mechanism, in some Mcl-1 dependent cancers, a full apoptotic response may require inhibition and/or depletion of more than one anti-apoptotic protein. Because inhibition of CDK9 can reduce the expression of other anti-apoptotic proteins in addition to Mcl-1 (such as, for example, Bfl-1), some Mcl-1 dependent cancers can be more sensitive to an inhibitor of CDK9 than to an Mcl-1 inhibitor.

The terms “treat,” “treating,” and “treatment” refer to at least partially alleviating, inhibiting, preventing and/or ameliorating a condition, disorder, or disease, such as cancer. The terms “treatment of cancer” or “treatment of cancer cells” include both in vitro and in vivo treatments, including in warm-blooded animals such as humans. The effectiveness of treatment of cancer cells can be assessed in a variety of ways, including but not limited to: inhibiting cancer cell proliferation (including the reversal of cancer growth); promoting cancer cell death (e.g., by promoting apoptosis or another cell death mechanism); improvement in symptoms; duration of response to the treatment; delay in progression of disease; and prolonging survival.

Treatments can also be assessed with regard to the nature and extent of side effects associated with the treatment. Furthermore, effectiveness can be assessed with regard to biomarkers, such as levels of expression or phosphorylation of proteins known to be associated with particular biological phenomena. Other assessments of effectiveness are known to those of skill in the art.

As used herein, an “Mcl-1 dependent cancer” refers to a cancer in which depletion or inhibition of Mcl-1 results in increased apoptosis of cancer cells sufficient to demonstrate a clinically beneficial effect. Apoptosis can be assessed by various means, such as cell death, increase in cleaved caspase, or other methods known in the art.

As used herein, “Bfl-1 positive” refers to cancers, cancer cells, or cancer cell lines that express Bfl-1 protein. In contrast, “Bfl-1 negative” refers to cancers, cancer cells, or cancer cell lines that do not express Bfl-1 protein. The status of Bfl-1 expression for a given cancer, cancer cell or cancer cell line can be determined, for example, by western blot.

As used herein, the term “an inhibitor of CDK9” refers to a compound that can inhibit CDK9, and, optionally, can inhibit one or more other CDKs. A compound that inhibits one or more other CDKs in addition to CDK9 is a non-selective inhibitor of CDK9, even if the primary target of the compound is not CDK9. For example, dinaciclib inhibits multiple CDKs, including CDK9. Thus, dinaciclib is a non-selective inhibitor of CDK9, as the term is used herein. A selective inhibitor of CDK9 is a compound that inhibits CDK9 and has little or no inhibitory activity toward other CDKs. Thus, “an inhibitor of CDK9” as used herein includes both non-selective and selective inhibitors of CDK9.

Inhibitors of CDK9 include, for example, AZD4573, BAY-1251152, BAY-1143572, CYC065, alvocidib, AT7519, voruciclib, roniciclib, and dinaciclib. Selective inhibitors of CDK9 include AZD4573, BAY-1251152, and BAY-1143572. Non-selective inhibitors of CDK9 include CYC065, alvocidib, AT7519, voruciclib, roniciclib, and dinaciclib.

AZD4573, a selective CDK9 inhibitor, also referred to as (1S,3R)-3-acetamido-N-(5-chloro-4-(5,5-dimethyl-5,6-dihydro-4H-pyrrolo [1,2-b]pyrazol-3-yl)pyridin-2-yl)cyclohexanecarboxamide, has the formula:

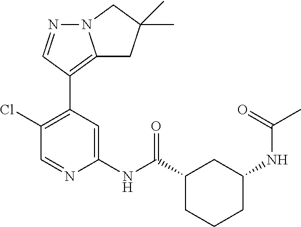 [SMILES: CC(=O)N[C@@H]1CCC[C@H](C(=O)Nc2cc(-c3cnn4c3CC(C)(C)C4)c(Cl)cn2)C1]

and is described in, for example, WO 2017/001354, which is incorporated by reference in its entirety.

As used herein, the term “Mcl-1 inhibitor” refers to a compound that can inhibit Mcl-1 by binding to Mcl-1. As used herein, the term “Mcl-1 inhibitor” excludes compounds that indirectly affect Mcl-1 by, for example, limiting expression of Mcl-1 protein. Thus, as the terms are used herein, an inhibitor of CDK9 would not be considered an Mcl-1 inhibitor. One illustrative example of an Mcl-1 inhibitor is AZD5991:

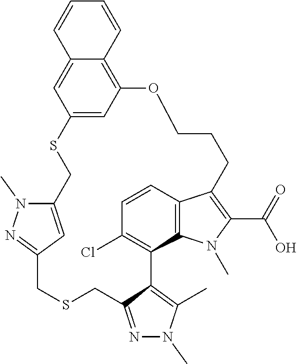

as described in U.S. Pat. No. 9,840,518, which is incorporated by reference in its entirety.

Cancer cell lines which are Mcl-1 dependent can vary in their sensitivity to treatments such as Mcl-1 inhibitors and inhibitors of CDK9. In a panel of Mcl-1 dependent cancer cell lines, it was unexpectedly found that Bfl-1 positive cell lines tended to show decreased sensitivity to Mcl-1 inhibition, increased sensitivity to CDK9 inhibition, or both, compared to Bfl-1 negative cell lines. Thus Bfl-1 positive cell lines were associated with greater relative sensitivity to an inhibitor of CDK9 compared to an inhibitor of Mcl-1. In this way, Bfl-1 can be used to distinguish among Mcl-1 dependent cancers to identify those that are likely to be sensitive to an inhibitor of CDK9, even if it is insensitive to an Mcl-1 inhibitor.

In one embodiment, a method of treating an Mcl-1 dependent cancer in a patient is provided, the method including determining whether the cancer is Bfl-1 positive by obtaining or having obtained a biological sample from the patient, and performing or having performed an assay to measure the expression level of Bfl-1; and if the cancer is Bfl-1 positive, then administering an inhibitor of CDK9 to the patient, thereby increasing cancer apoptosis; where the increase in cancer apoptosis for a Bfl-1 positive cancer upon administration of an inhibitor of CDK9 is greater than it would be in a Bfl-1 negative cancer, and/or the increase in cancer apoptosis for a Bfl-1 positive cancer upon administration of an Mcl-1 inhibitor is less than it would be in a Bfl-1 negative cancer.

In one embodiment, a method of increasing cancer apoptosis in an Mcl-1 dependent cancer is provided, the method including determining whether the cancer is Bfl-1 positive by obtaining or having obtained a biological sample from the patient, and performing or having performed an assay to measure the expression level of Bfl-1; and if the cancer is Bfl-1 positive, then administering an inhibitor of CDK9 to the patient, thereby increasing cancer apoptosis; where the increase in cancer apoptosis for a Bfl-1 positive cancer upon administration of an inhibitor of CDK9 is greater than it would be in a Bfl-1 negative cancer, and/or the increase in cancer apoptosis for a Bfl-1 positive cancer upon administration of an Mcl-1 inhibitor is less than it would be in a Bfl-1 negative cancer.

In one embodiment, a method of reducing levels of one or more anti-apoptotic proteins in an Mcl-1 dependent cancer is provided, the method including determining whether the cancer is Bfl-1 positive by obtaining or having obtained a biological sample from the patient, and performing or having performed an assay to measure the expression level of Bfl-1; and if the cancer is Bfl-1 positive, then administering an inhibitor of CDK9 to the patient, thereby increasing cancer apoptosis; where the increase in cancer apoptosis for a Bfl-1 positive cancer upon administration of an inhibitor of CDK9 is greater than it would be in a Bfl-1 negative cancer, and/or the increase in cancer apoptosis for a Bfl-1 positive cancer upon administration of an Mcl-1 inhibitor is less than it would be in a Bfl-1 negative cancer.

In each of the above embodiments, the method can further include administering an Mcl-1 inhibitor to the patient if the cancer is Bfl-1 negative. The inhibitor of CDK9 can be a selective inhibitor of CDK9. The inhibitor of CDK9 can be AZD4573. The cancer can be selected from diffuse large B-cell lymphoma (DLBCL), mantle cell lymphoma, chronic lymphocytic leukemia, small chronic lymphocytic leukemia, Waldentröm's macroglobulinemia, marginal zone lymphoma, chronic graft versus host disease, follicular lymphoma, and acute lymphoblastic leukemia. As used herein, DLBCL includes activated B-cell DLBCL (ABC-DLBCL) and germinal center B-cell DLBCL (GCB-DLBCL). The cancer can be a lymphoma. The cancer can be diffuse large B-cell lymphoma (DLBCL). The cancer can be activated B-cell diffuse large B-cell lymphoma (ABC-DLBCL). The Mcl-1 inhibitor can be AZD5991.

In one embodiment, the use of an inhibitor of CDK9 in the treatment of an Mcl-1 dependent cancer, is provided where the cancer has been determined to be Bfl-1 positive. The inhibitor of CDK9 can be a selective inhibitor of CDK9. The inhibitor of CDK9 can be AZD4573.

In one embodiment, the use of an Mcl-1 inhibitor in the treatment of an Mcl-1 dependent cancer, is provided where the cancer has been determined to be Bfl-1 negative. The Mcl-1 inhibitor can be AZD5991.

In one embodiment, a method of treating lymphoma in a patient is provided, the method including determining whether the lymphoma is Bfl-1 positive by obtaining or having obtained a biological sample from the patient, and performing or having performed an assay to measure the expression level of Bfl-1; and if the lymphoma is Bfl-1 positive, then administering an inhibitor of CDK9 to the patient, thereby increasing cancer apoptosis; where the increase in cancer apoptosis for a Bfl-1 positive lymphoma upon administration of an inhibitor of CDK9 is greater than it would be in a Bfl-1 negative lymphoma, and/or the increase in cancer apoptosis for a Bfl-1 positive lymphoma upon administration of an Mcl-1 inhibitor is less than it would be in a Bfl-1 negative lymphoma.

In one embodiment, a method of treating activated B-cell diffuse large B-cell lymphoma (ABC-DLBCL) in a patient is provided, the method including determining whether the lymphoma is Bfl-1 positive by obtaining or having obtained a biological sample from the patient, and performing or having performed an assay to measure the expression level of Bfl-1; and if the lymphoma is Bfl-1 positive, then administering an inhibitor of CDK9 to the patient, thereby increasing cancer apoptosis; where the increase in cancer apoptosis for a Bfl-1 positive lymphoma upon administration of an inhibitor of CDK9 is greater than it would be in a Bfl-1 negative lymphoma, and/or the increase in cancer apoptosis for a Bfl-1 positive lymphoma upon administration of an Mcl-1 inhibitor is less than it would be in a Bfl-1 negative lymphoma.

### EXAMPLE 1: A SUBSET OF LYMPHOMA TUMOR MODELS SHOW ENHANCED SENSITIVITY TO CDK9 INHIBITION COMPARED TO MCL-1 INHIBITION

A subset of lymphoma cell lines displays enhanced sensitivity to CDK9 inhibition compared to Mcl-1 inhibition. In a 6-hour caspase activation assay, 33 lymphoma cell lines (identified in FIG. 2A) were treated with the selective CDK9 inhibitor, AZD4573, or the selective Mcl-1 inhibitor, AZD5991. Seven of the thirty-three cell lines displayed a greater than 20-fold enhanced sensitivity to CDK9 inhibition with AZD4573 treatment compared to AZD5991 based on caspase EC₅₀. Additionally, the magnitude of caspase activation in a subset of these models was significantly greater in response to CDK9 inhibition compared to Mcl-1 inhibition.

Method: 33 diffuse large B-cell (DLBCL) and Mantle cell (MCL) lymphoma cell lines were seeded onto 384 well plates pre-dosed with 10-point, ½ log titrations of AZD4573 or AZD5991, and incubated for 6 hours. Following the incubation cleaved caspase was measured using Caspase-Glo 3/7 (Promega) following the manufacturer's protocol. Data was analyzed using GeneData Screener and Spotfire.

Results are shown in FIGS. 1A-1B: Pharmacological response of lymphoma tumor models to CDK9 and Mcl-1 inhibitors. FIG. 1A is a scatterplot comparing AZD4573 and AZD5991 caspase pEC₅₀ values across a 33 cell line panel. Solid and dashed lines display 1:1 or 1:10 y=x axis unity, respectively. Cell lines highlighted in red/indicated by arrows have >20× more potent shift in caspase EC₅₀ in response to AZD4573 treatment compared to AZD5991 treatment. FIG. 1B is a scatterplot correlating AZD4573 and AZD5991 caspase maximum effect following a 6 hour compound incubation. Cell lines highlighted in red/indicated by arrows fit criteria of >50% max effect in response to AZD4573 treatment AND <50% max effect in response to AZD5991 treatment.

### EXAMPLE 2: BFL-1 EXPRESSING LYMPHOMA TUMOR MODELS ARE HIGHLY SENSITIVE TO CDK9 INHIBITION BUT LESS SENSITIVE TO MCL-1 INHIBITION

Protein expression of the Bcl2 family members was evaluated to help understand why a subset of lymphoma models have enhanced sensitivity to CDK9 inhibition compared to Mcl-1 inhibition. Bfl-1 expression was identified in over 20% of lymphoma cell lines evaluated (n=33), with the majority of expressing cell lines belonging to the ABC-DLBCL lymphoma subtype. When cell lines were clustered based on no expression of Bfl-1 (n=25), the geometric mean caspase EC₅₀ difference between AZD5991 and AZD4573 was only 4-fold. Interestingly, there was a dramatically larger 22-fold difference between AZD5991 and AZD4573 caspase EC₅₀'s in Bfl-1 expressing cell lines (n=8). A similar trend was observed when also applying the median caspase EC₅₀.

Method: Cell lysates from 33 lymphoma cell lines were generated in parallel to evaluate protein expression of pro-survival Bcl2 family members. Cell lysates were normalized for protein concentration using the BCA Protein Assay Kit, and western blots were run according to standard protocols. A positive cell line control for expression of Bfl-1, TMD8, was utilized for normalization to other cell lines in the panel. AZD4573 and AZD5991 respective median and geomean caspase EC₅₀'s were calculated from cell lines clustered based on high and low Bfl-1 expression, respectively.

Results are shown in FIGS. 2A-2B: Bfl-1 protein expression across lymphoma tumor models. FIG. 2A shows evaluation of Bfl-1 protein expression in 33 lymphoma cell lines. Bar colors represent lymphoma subtype. Bars crossing through the dotted line were positive for Bfl-1 expression by western blot. The scale is relative to expression of the TMD8 cell line control. FIG. 2B shows caspase geomean and median EC₅₀'s for AZD4573 and AZD5991 across the 33 lymphoma tumor models. Tables are grouped into clusters based on positive or negative Bfl-1 expression. The EC₅₀ fold difference between AZD5991 and AZD4573 is listed for each cell line category.

### EXAMPLE 3: BFL-1 IS A LABILE PROTEIN MODULATED BY TRANSIENT AZD4573 TREATMENT IN LYMPHOMA CELL LINES

Since a positive correlation was found between Bfl-1 expression and enhanced sensitivity to CDK9 inhibition, it was hypothesized that CDK9 inhibition may be targeting Bfl-1 in addition to Mcl-1 in lymphoma cell lines. The ABC-DLBCL cell line OCILY10 was treated with serial-dilutions of AZD4573 and immunoblotted for downstream target proteins. The proximal CDK9 biomarker, pSer2-RNAP2, was inhibited in a dose-dependent manner with AZD4573. Equipotent reductions in Mcl-1 and Bfl-1 protein were observed at concentrations of AZD4573 that inhibited the proximal CDK9 biomarker. Bfl-1 was confirmed to be a labile protein by treating OCILY10 cells with cycloheximide, revealing Bfl-1 has a half-life of less than 1 hour, similar to Mcl-1. The half-life of other Bcl2 family proteins were all greater than 9 hours.

A time-dependent response to CDK9 inhibition was also observed in OCILY10 cells treated with 100 nM AZD4573. Expression of pSer2-RNAP2 was inhibited >80% 30 minutes after dosing the cells, whereas Mcl1 and Bfl1 mRNA expression were reduced to equivalent levels by 2 hours, followed by reductions in Mcl-1 and Bfl-1 protein at 4 hours. Cleaved caspase was detected at 6 hours. Expression of longer lived proteins Bcl2, Bcl-xL and Bcl-W remained relatively unchanged after 6 h treatment with AZD4573. We observed similar results of Bfl-1 and Mcl-1 following AZD4573 treatment in an additional ABC-DLBCL cell line, TMD8.

Method: The ABC-DLBCL cell line OCILY10 was treated for 6 h with a 9 pt, ½ log dose response of AZD4573 and cells were harvested for protein lysates. These cells were also treated with AZD4573 at 100 nM for varying timepoints out to 6 hours. At each timepoint (0, 0.5, 1, 2, 4, 6 hours) cells were harvested for either mRNA isolation, or protein lysates, respectively. mRNA was converted to cDNA and PCR sequence amplification was performed with Mcl1 and Bcl2a1 primers following standard protocols. Protein lysates were normalized for protein concentration using the BCA Protein Assay Kit, and run for western blots according to standard protocols. To ensure expected target engagement was achieved, the blots were probed for the proximal biomarker for CDK9 (pSer2-RNAPolII), along with Mcl-1 and Bfl-1. To gauge the time to induction of apoptosis, cleaved caspase-3 was assessed. A loading control (vinculin) was also utilized for normalization.

Cell lysates from thirty-three DLBCL and MCL lymphoma cell lines were generated in parallel to evaluate protein expression of pro-survival Bcl2 family members. Cell lysates were normalized for protein concentration using the BCA Protein Assay Kit, and western blots were run according to standard protocols. Controls were utilized to ensure equal loading of protein (GAPDH) and equal expression across western blot assays using a positive control cell lysates from TMD8 cells.

To estimate the half-life of Bcl2 family proteins, OCILY10 cells were treated with 10 μg/mL of cycloheximide to arrest new protein synthesis. 1e\hat{ }6 cells were harvested for generating protein lysates at varying time points (0, 1, 3, 6, 9, 24 hours) post-cycloheximide treatment.

Results are shown in FIGS. 3A-3D. Inhibition of Bfl-1 by AZD4573 treatment in lymphoma cell lines: FIG. 3A: OCILY10 cells treated for 6 hours with a dose-response of AZD4573 and evaluated by western blot. FIG. 3B: OCILY10 cells were treated with 10 μg/mL cycloheximide for the indicated timepoints and immunoblotted for Bcl2 family protein expression. FIG. 3C: Kinetics of Bfl-1 transcript and protein modulation in OCILY10 cells over time following treatment with 100 nM of AZD4573. FIG. 3D: Immunoblot data from TMD8 cells treated with 100 nM of AZD4573 for the indicated timepoints and evaluated for protein modulation of Mcl-1, Bfl-1, and cleaved caspase.

### EXAMPLE 4: BFL-1 EXPRESSING LYMPHOMA CELL LINES DEPEND ON MULTIPLE BCL-2 FAMILY PROTEINS FOR SURVIVAL

The single-gene dependency of lymphoma cells on Bfl-1 using siRNA was determined as described below. Upon >80% knockdown with siRNA targeting Bfl-1 in OCILY10 and TMD8 cell lines, expression of the intrinsic apoptosis biomarker cleaved PARP did not increase relative to the scrambled control. However, when Bfl-1 knockdown cells were treated with AZD5991 and reassessed for cleaved caspase, the maximum level of apoptosis achieved in both OCILY10 and TMD8 cell lines increased from an average of 45% to over 90%, phenocopying a similar magnitude of maximum caspase activation achieved in response to transient CDK9 inhibition via AZD4573.

Method: OCILY10 and TMD8 cells were grown in logarithmic phase and plated in serum-free siRNA delivery media at 1×10\hat{ }6 cells/mL in 12-well plates (Dharmacon B-005000). An siRNA SMARTpool targeting Bfl-1 or a scrambled negative control (NTC) were added to respective wells for 24 hours at 0.1 or 0.5 μM (Dharmacon Bcl2a1-597, NTC-D-001910-01). 2 mL of cell culture media was added to all transfected wells and transferred into a 6-well plate for an additional 48 hours. Transfected cells were collected to assess Bfl-1 protein knockdown and cleaved caspase-3 by western blot. Transfected cells were also seeded onto 384 well plates pre-dosed with an 8-point titration of AZD4573 or AZD5991 and incubated for 6 hours. The plates were measured for activation of cleaved caspase using Caspase-Glo 3/7 (Promega) following the manufacturer's protocol.

Results are shown in FIGS. 4A-4B. Bfl-1 dependency in lymphoma models: FIG. 4A: Immunoblot showing Bfl-1 and cleaved PARP expression in OCILY10 and TMD8 cell lysates after transfection of Bfl-1 siRNA. FIG. 4B: Graphs showing a dose-dependent increase in caspase activation in OCILY10 and TMD8 cell lines for AZD5991 treatment under Bfl-1 knockdown conditions.

### EXAMPLE 5: AZD4573 IN ABC-DLBCL CELL LINES DEMONSTRATES ROBUST ANTI-TUMOR ACTIVITY

Effects of CDK9 inhibition on Bfl-1 expression in vivo were evaluated. Intermittent dosing of the ABC-DLBCL xenografts OCILY10 and TMD8 with AZD4573 caused robust tumor regressions (198 and 184% TGI, respectively), compared to Mcl-1 inhibition. AZD4573-mediated anti-tumor activity was associated with pharmacodynamic reductions of pSer2-RNAPII, Mcl-1 and Bfl-1, followed by caspase activation.

Method: AZD4573 was formulated in dimethylacetamide (DMA)/polyethylene glycol 400 (PEG 400)/1% w/v Tween 80 solution 2/30/68 and dosed at 15 mg/kg, intraperitoneally (ip), BID with a 2 hour split on days 1 and 2 with a 5 day dose holiday.

AZD5991 was formulated for intravenous use in 30% HPBCD (hydroxy-propyl-beta-cyclodextrin) in water-for-injection adjusted to pH 9.0-9.5 up to a concentration of 20 mg/mL (based on parent form). AZD5991 was dosed at 60 mg/kg by tail-vain intravenous injection once weekly.

5×10⁶ OCILy10 tumor cells or 10×10⁶ TMD8 tumor cells were injected subcutaneously in the right flank of C.B.-17 SCID female mice in a volume of 0.1 mL containing 50% matrigel.

Tumor volumes (measured by caliper), animal body weight, and tumor condition were recorded twice weekly for the duration of the studies. The tumor volume was calculated using the formula: length (mm)×width (mm)²×0.52. For efficacy studies, growth inhibition from the start of treatment was assessed by comparison of the differences in tumor volume between control and treated groups. Dosing began when mean tumor size reached approximately 150-180 mm³. CR=complete response.

Pharmacodynamic measurements were performed following standard protocols for tumor dissociation. Protein lysates from dissociated tumors were normalized using the BCA protein assay kit. Immunoblots were run and probed for expression of pSERII-RNAPII and Bcl2 family proteins. A loading control (GAPDH) was used to confirm equal protein loading.

Results are shown in FIGS. 5A-5B: in vivo anti-tumor activity of AZD4573 in Bfl-1 expression lymphoma xenografts. FIG. 5A: AZD4573 treatment leads to complete tumor regressions in the OCILY10 and TMD8 ABC-DLBCL xenograft models. FIG. 5B: Summary table of AZD4573 and AZD5991 efficacy achieved, respectively, after 3 dosing cycles. FIG. 5C: Pharmacodynamic modulation of Bfl-1 following acute AZD4573 treatment in OCILY10 and TMD8 models.

Other embodiments are within the scope of the following claims.

What is claimed is:

1. A method of treating an Mcl-1 dependent cancer in a patient, comprising:
determining whether the cancer is Bfl-1 positive by:
obtaining or having obtained a biological sample from the patient; and
performing or having performed an assay to measure the expression level of Bfl-1; and
if the cancer is Bfl-1 positive, then administering an inhibitor of CDK9 to the patient, thereby increasing cancer apoptosis;
wherein the increase in cancer apoptosis for a Bfl-1 positive cancer upon administration of an inhibitor of CDK9 is greater than it would be in a Bfl-1 negative cancer, and/or the increase in cancer apoptosis for a Bfl-1 positive cancer upon administration of an Mcl-1 inhibitor is less than it would be in a Bfl-1 negative cancer.

2. The method of claim 1, further comprising administering an Mcl-1 inhibitor to the patient if the cancer is Bfl-1 negative.

3. The method of any one of claims 1 to 2, wherein the inhibitor of CDK9 is a selective inhibitor of CDK9.

4. The method of any one of claims 1 to 3, wherein the inhibitor of CDK9 is AZD4573.

5. The method of any one of claims 1 to 4, wherein the cancer is a lymphoma.

6. The method of claim 5, wherein the cancer is diffuse large B-cell lymphoma (DLBCL).

7. The method of claim 6, wherein the cancer is activated B-cell diffuse large B-cell lymphoma (ABC-DLBCL).

8. The method of any one of claims 2 to 7, wherein the Mcl-1 inhibitor is AZD5991.

9. Use of an inhibitor of CDK9 in the treatment of an Mcl-1 dependent cancer, wherein the cancer has been determined to be Bfl-1 positive.

10. The use of claim 9, wherein the inhibitor of CDK9 is a selective inhibitor of CDK9.

11. The use of any one of claims 9 to 10, wherein the inhibitor of CDK9 is AZD4573.

12. Use of an Mcl-1 inhibitor in the treatment of an Mcl-1 dependent cancer, wherein the cancer has been determined to be Bfl-1 negative.

13. The use of claim 13, wherein the Mcl-1 inhibitor is AZD5991.
